(19) United States
(12) Patent Application Publication
(10) Pub. No.: US 2009/0197907 A1
(43) Pub. Date: Aug. 6, 2009

(54) NOVEL CRYSTALLINE FORM OF RUPATADINE FREE BASE

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Kesireddy Subash Chander Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad Andhrapradesh (IN)

(21) Appl. No.: 10/598,846

(22) PCT Filed: Apr. 1, 2005
(86) PCT No.: PCT/IN2005/000097

§ 371 Date: Mar. 24, 2009

(51) Int. Cl. C07D 221/06; A61K 31/44

(52) U.S. Cl. 514/290; 546/93

(57) ABSTRACT

The present invention relates to a novel crystalline form of rupatadine free base, process for its preparation and to a pharmaceutical composition containing it. In accordance with the present invention rupatadine is suspended in n-hexane, n-heptane, cyclohexane, diethyl ether or diisopropyl ether, stirred for at least 1 hour, filtered the solid and dried to give crystalline rupatadine form-B. The isolation of novel rupatadine free base as crystalline form-B may be useful as a purification of rupatadine or a salt thereof.

### FIELD OF THE INVENTION

The present invention relates to a novel crystalline form of rupatadine free base, to a process for its preparation and to a pharmaceutical composition containing it.

### BACKGROUND OF THE INVENTION

EP 577957 disclosed a series of 3-pyridylmethyl derivatives of 8-chloro-11-(4-pyperidyliden)-6,11-dihydro-5H-benzo[5,6]cyclohepta[1,2-b]pyridine, among them rupatadine, chemically 8-chloro-11-[1-[(5-methyl-3-pyridyl)methyl]piperidin-4-ylidene]6,11-dihydro-5H-benzo[5,6]cyclohepta[1,2-b]pyridine is a potent platelet activating factor (PAF) antagonist and useful in the treatment of the diseases in which PAF and/or histamine are involved. Rupatadine is represented by the following structure:

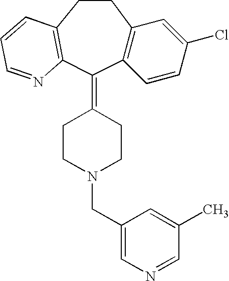

The synthesis and therapeutic uses of rupatadine are disclosed in EP 577957, U.S. Pat. No. 5,407,941 and Journal of Medicinal Chemistry 1994, 37(17), 2697-2703. These patents and journal document are incorporated herein by reference.

Rupatadine can be obtained in a number of methods. Thus, for example, 8-chloro-11-(4-piperidyliden)-6,11-dihydro-5H-benzo[5,6]cyclohepta[1,2-b]pyridine of formula II:

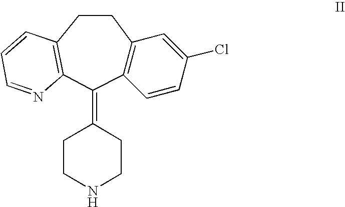

is reacted with 3-bromomethyl-5-methylpyridine of formula III:

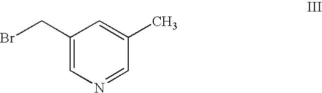

to obtain rupatadine of formula I:

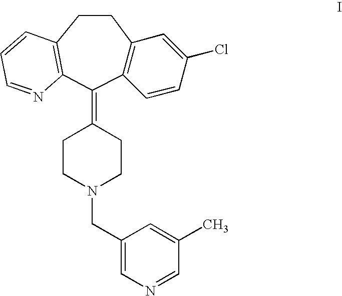

U.S. Pat. No. 5,407,941 described that rupatadine obtained (in example 4) is purified using column chromatography and the rupatadine free base obtained in the process has melting point of 58°-61° C.

Journal of Medicinal Chemistry 1994, 37(17), 2697-2703 described that rupatadine obtained (page No. 2701) is purified using column chromatography and the rupatadine free base obtained in the process has melting point of 58°-61° C.

It has now been found that rupatadine free base can be obtained in another crystalline form designated as crystalline form-B. For the sake of convenience, rupatadine crystalline form obtained in, for example, U.S. Pat. No. 5,407,941, is designated as crystalline form-A.

It has also been found that no chromatographic purification is required if rupatadine free base can be isolated as crystalline form-B from reaction mass comprising rupatadine.

It has also been found that isolation method of rupatadine free base as crystalline form-B can be used for purification of impure rupatadine free base or a salt thereof.

### DETAILED DESCRIPTION OF THE INVENTION

In accordance with the present invention, there is provided a novel crystalline form of rupatadine free base, designated as form-B, characterized by having the melting point of about 110-115° C. The melting point was measured on Polmon make MP96 melting apparatus.

The crystalline rupatadine form-B is further characterized by a differential scanning calorimetric (pSC) thermogram with endothermic peak at about 112° C. as shown in FIG. 1.

The crystalline rupatadine form-B is further characterized by an x-ray powder diffraction (X-RD) spectrum having peaks expressed as 2θ at about 18.2, 18.5, 18.8, 19.5, 20.2, 22.7 and 23.8 degrees. FIG. 2 shows a typical form-B x-ray powder diffraction spectrum.

The crystalline rupatadine form-B is further characterized by an x-ray powder diffraction (X-RD) spectrum having peaks expressed as 2θ at about 9.4, 9.8, 14.9, 16.4, 18.2, 18.5, 18.8, 19.5, 20.2, 22.7, 23.8, 24.5 and 28.4 degrees. FIG. 2 shows a typical form-B x-ray powder diffraction spectrum.

The crystalline rupatadine form-B is further characterized by a Fourier transform Infrared (FTIR) spectrum as shown in FIG. 3.

In accordance with the present invention, a process is provided for preparation of crystalline rupatadine form-B, which comprises suspending rupatadine in n-hexane, n-heptane, cyclohexane, diethyl ether or diisopropyl ether, stirring for at least about one hour and isolating rupatadine free base as crystalline form-B.

The stirring of the suspension is carried out preferably for 1 to 10 hours, more preferably for 3-6 hours at below the boiling temperature of the solvent used, preferably at 15° C. to the boiling temperature of the solvent used and comfortably at ambient temperature.

The isolation of the crystalline form-B may be carried out by usually known methods such as filtration or centrifugation.

The starting material, rupatadine may be obtained as a reaction mass obtained by, for example, reaction of 8-chloro-11-(4-piperidyliden)-6,11-dihydro-5H-benzo[5,6]cyclohepta[1,2-b]pyridine with 3-bromomethyl-5-methylpyridine. In such a case of using reaction mass as the starting material, rupatadine free base is obtained as crystalline form-B in substantially pure form without the need for chromatographic technique.

In the case where further purification of rupatadine free base is required such a material may be used as the starting material.

In the case where further purification of a salt of rupatadine is required, the rupatadine salt is first reacted with a base to obtain rupatadine free base and then crude rupatadine is isolated from the reaction mass and the crude rupatadine obtained may be used as a starting material of the invention.

The above said process serves the purposes of method of preparing crystalline form-B and methods of purifications.

The crystalline form-B is obtained in a High Performance Liquid Chromatography (HPLC) purity of about 95% or above, usually above about 98%.

The rupatadine crystalline form-B is stable, can be obtained in high purity and is useful in the treatment of the diseases in which PAF and/or histamine are involved; and so, the novel form may be used in the pharmaceutical preparations for treating diseases in which PAF and/or histamine are involved.

### BRIEF DESCRIPTION OF THE DRAWINGS

FIG. 1 shows the Differential Scanning Calorimetric thermogram of crystalline rupatadine form-B.

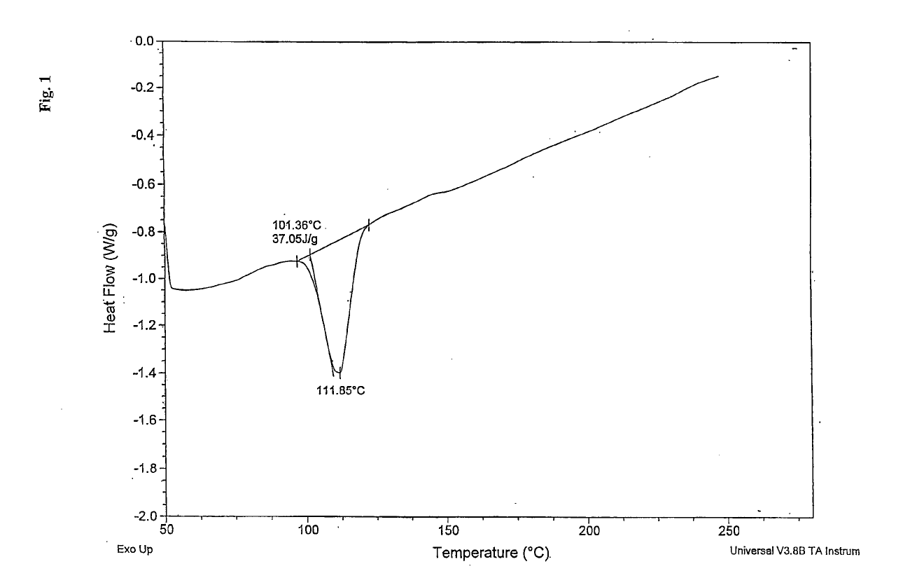

FIG. 2 shows the X-ray diffraction diagram of crystalline rupatadine form-B.

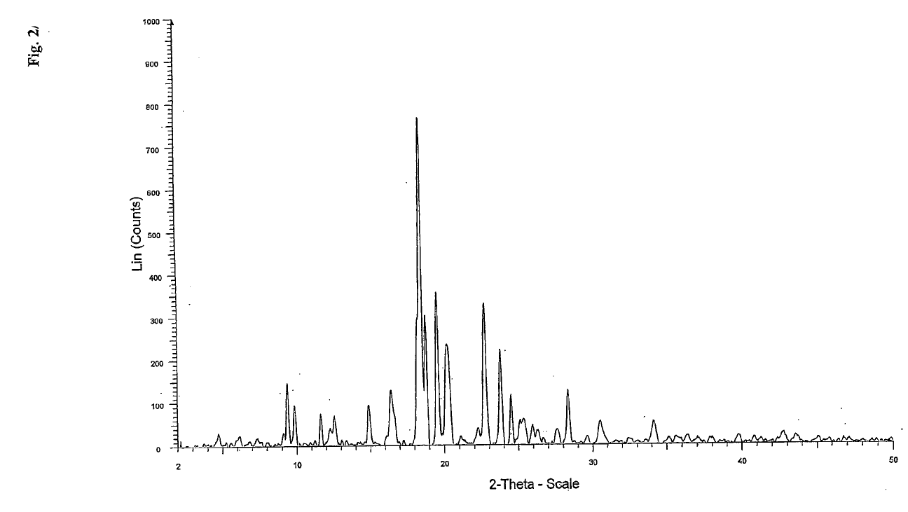

FIG. 3 shows the FTIR spectrum of crystalline rupatadine form-B.

DSC (Differential Scanning Calorimetry) measurements were performed with a bSC Q10 (TA Instruments, Inc.). About 3 mg of the powder was placed in an open aluminum pan and it is crimped with an aluminum lid. The crimped sample is then placed in the DSC cell opposite to empty aluminum pan (as reference) and the sample was scanned at 10° C./min from 50° C. to 280° C.

x-Ray powder diffraction spectrum was measured on a Bruker axs D8 advance x-ray powder diffractometer having a Copper-Kα radiation. Approximately 1 gm of sample was gently flattened on a sample holder and scanned from 2 to 50 degrees two-theta, at 0.03 degrees two-theta per step and a step time of 0.5 seconds. The sample was simply placed on the sample holder. The sample was rotated at 30 rpm at a voltage 40 KV and current 35 mA.

FT-IR spectroscopy was carried out with a Perkin-Elmer spectrum GX spectrometer. For the production of the KBr compacts approximately 2 mg of sample was powdered with 200 mg of KBr. The spectra were recorded in transmission mode ranging from 4000 to 400 cm⁻¹.

The following examples are given for the purpose of illustrating the present invention and should not be considered as limitations on the scope or spirit of the invention.

### EXAMPLE 1

a) Ethanol (100 ml) is added to 11-[N-(ethoxycarbonyl)-4-piperidylidene]-8-chloro-6,11-dihydro-5H-benzo-[5,6]cyclohepta[1,2-b]pyridine (15 gm) and then potassium hydroxide solution (22.5 gm of KOH in 90 ml of water) is added for 20 minutes at 25-40° C. The contents are heated to reflux, stirred for 22 hours and distilled off ethanol under vacuum below 50° C. To the aqueous part is added sodium chloride (15 gm), extracted three times with ethyl acetate (each time 50 ml), dried and distilled off the ethyl acetate layer. Acetonitrile (20 ml) is added to the residual solid and stirred for 30 minutes at 10-15° C. Filter the solid, washed with 5 ml of acetonitrile and dried to give 10.5 gm of 8-Chloro-11-(4-piperidyliden)-6,11-dihydro-5H-benzo[5,6]cyclohepta[1,2-b]pyridine (HPLC purity: 99.5%).
b) N-Bromosuccinamide (30 gm) and 2,2′-azobisisobutyronitrile (1 gm) are added to a solution of 3,5-lutidine (17 ml) in 200 ml of carbontetrachloride, heated to 75° C. and stirred for 1 hour 30 minutes at 75-80° C. The reaction mass is cooled to 25° C. and filtered the cake. To the filtrate is added 8-Chloro-11-(4-piperidyliden)-6,11-dihydro-5H-benzo[5,6]cyclohepta[1,2-b]pyridine (10 gm) obtained in (a) above, chloroform (100 ml), triethylamine (17 gm) and dimethylaminopyridine (0.5 gm), and stirred for 18 hours at 25-30° C. Then 5% NaHCO₃ solution (300 ml) is added to the reaction mass, stirred for 15 minutes, separated the layers and the organic layer is washed two times with water (each time 300 ml). The organic layer is separated and distilled under vacuum below 50° C. to give 15 gm of rupatadine as a residue (HPLC purity: 86.0%).

The residue is suspended in with cyclohexane (50 ml), stirred for 6 hours at 15-25° C., filtered the solid and dried at 40-50° C. to give 6.5 gm of crystalline rupatadine form-B (HPLC purity: 99.3%).

### EXAMPLE 2

Fumaric acid (1.7 gm) is added to the solution of crystalline rupatadine form-B (5 gm) in ethanol (40 ml), stirred for 15 minutes at 75-80° C. and then cooled to 25° C. The contents are stirred for 10 hours at 25-30° C., filtered the solid, washed with ethanol (10 ml) and dried at 40-50° C. to give 5 gm of rupatadine fumarate (HPLC purity: 99.5%, characterized by an x-ray powder diffraction (X-RD) spectrum having peaks expressed as 20 at about 8.2, 11.9, 12.7, 13.8, 16.3, 16.8, 19.8, 20.4, 22.4, 23.2, 23.8, 24.6 and 27.0 degrees).

### EXAMPLE 3

Rupatadine (10 gm, HPLC purity: 94.1%) is suspended in n-hexane (50 ml), stirred for 5 hours at 15-25° C., filtered the solid and dried at 40-50° C. to give 8.5 gm of crystalline rupatadine form-B (HPLC purity: 99.9%).

### EXAMPLE 4

Water (100 ml) and methylene dichloride (100 ml) are added to rupatadine fumarate (HPLC purity: 94.2%), pH is adjusted to 8.5 with sodium hydroxide. The layers are separated and distilled the organic layer under vacuum at below 40° C. To the residue is added n-hexane (35 ml), stirred for 5 hours at 15-20° C., filtered the solid and dried at 40-50° C. to give 5.5 gm of crystalline rupatadine form-B (HPLC purity: 99.8%).

We claim:

1. A crystalline rupatadine form-B characterized by having the melting point of about 110-115° C.

2. The crystalline rupatadine form-B as defined in claim 1, further characterized by a differential scanning calorimetric thermogram with endothermic peak at about 112° C.

3. The crystalline rupatadine form-B as defined in claim 1, further characterized by an x-ray powder diffraction spectrum having peaks expressed as 2θ at about 18.2, 18.5, 18.8, 19.5, 20.2, 22.7 and 23.8 degrees.

4. The crystalline rupatadine form-B as defined in claim 1, further characterized by an x-ray powder diffraction spectrum having peaks expressed as 2θ at about 9.4, 9.8, 14.9, 16.4, 18.2, 18.5, 18.8, 19.5, 20.2, 22.7, 23.8, 24.5 and 28.4 degrees.

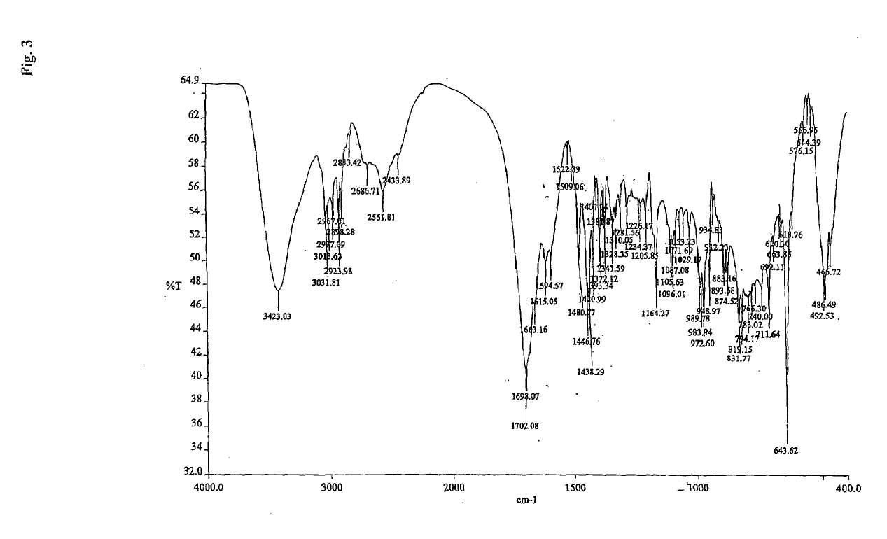

5. The crystalline rupatadine form-B as defined in claim 1, further characterized by a Fourier transform Infrared (FTIR) spectrum as shown in FIG. 3.

6. A process for preparation of crystalline rupatadine form-B as defined in claim 1, which comprises suspending rupatadine in n-hexane, n-heptane, cyclohexane, diethyl ether or diisopropyl ether, stirring for at least about one hour and isolating rupatadine free base as crystalline form-B.

7. The process according to claim 6, wherein the stirring of the suspension is carried out for 1 to 10 hours at below the boiling temperature of the solvent used.

8. The process according to claim 7, wherein the stirring of the suspension is carried out for 3-6 hours at 15° C. to the boiling temperature of the solvent used.

9. The process according to claim 8, wherein the stirring of the suspension is carried out for 3-6 hours at ambient temperature.

10. The process according to claim 6, wherein the isolation of the crystalline form-B is carried out by filtration or centrifugation.

11. A pharmaceutical composition comprising crystalline rupatadine form-B as defined in claim 1 and a pharmaceutically acceptable carrier or diluent.
